(19) United States
(12) Patent Application Publication
(10) Pub. No.: US 2022/0008629 A1
(43) Pub. Date: Jan. 13, 2022

(54) NON-BIODEGRADABLE ANTI-ADHESION MATERIAL

(71) Applicant: HI-LEX CORPORATION, Takarazuka-shi, Hyogo (JP)

(72) Inventor: Yasuharu Noishiki, Yokohama (JP)

(21) Appl. No.: 17/289,083

(22) PCT Filed: Oct. 21, 2019
(86) PCT No.: PCT/JP2019/041248

§ 371 Date: Apr. 27, 2021

(30) Foreign Application Priority Data
Oct. 31, 2018 (JP) 2018-205802

(51) Int. Cl. A61L 31/04; A61B 90/00

(52) U.S. Cl. CPC A61L 31/041; A61L 2300/424; A61B 90/02

(57) ABSTRACT

The purpose of the present invention is to inhibit and prevent the formation of adhesions, i.e., bonds between a wound and its surrounding tissues or between organs that are originally separated.

The anti-adhesion material of the present invention is characterized in that at least a part of the anti-adhesion material is made of a non-biodegradable material and a contact angle of a surface to water is less than 7 degrees or more than 90 degrees.

### TECHNICAL FIELD

The present invention relates to an anti-adhesion material that can inhibit and prevent the formation of adhesion, i.e., bonds between a wound and its surrounding tissue, or between organs that are normally separated.

### BACKGROUND ART

Surgical procedures can cause unexpected connections, or adhesions, between organs that should be separated (see, for example, non-patent document 1). Such adhesions can cause serious problems such as postoperative intestinal obstruction. In addition, when reoperation is required, if adhesions have occurred in the previous operation, the operation must be started by detaching the adhesions, which imposes a heavy burden on medical personnel and patients. Therefore, prevention of postoperative adhesions is an important issue in the medical field, and safe and reliable measures to prevent adhesions are desired.

Under these circumstances, various measures have been taken, such as devising surgical techniques, postoperative administration of adjuvant drugs, and use of in vivo biodegradable and absorbable anti-adhesion materials. Among these measures, the administration of adjuvant drugs and the use of anti-adhesion materials are expected to play a role as effective adjunctive measures.

However, the administration of adjuvant drugs has several problems such as (1) it is unclear whether there is an anti-adhesion effect or not, (2) delayed wound healing may occur, and (3) administration of drugs may cause further adhesion. For these reasons, the technological development of adjuvant drugs is practically stagnant.

In contrast, biodegradable and absorbable anti-adhesion materials have already been used in clinical practice. For example, a representative of commercially available bioabsorbable anti-adhesion membranes is the anti-adhesion material produced by Genzyme Corporation. It consists of a polyanionic hydrophilic biodegradable polymer obtained by cross-linking hyaluronic acid and carboxymethyl cellulose (CMC) with a carbodiimide compound, and is marketed under the name Seprafilm (registered trademark). This anti-adhesion material is a product designed to prevent post-operative adhesions in the abdominal and gynecological areas. This anti-adhesion material has been observed to have an effective anti-adhesion effect in organs such as the abdomen, where peristalsis occurs. However, as far as the data shows, its anti-adhesion effectiveness is about 50%, and it does not demonstrate anti-adhesion effectiveness in the thoracic region.

Conventional anti-adhesion materials are known to exist in three major types as follows:

(1) Those that are inserted as a physical barrier to prevent adhesions.

(2) The material itself has the ability to exclude cells, thereby preventing adhesion.

(3) Prevents adhesion by using substances that are effective in preventing adhesion.

The anti-adhesion materials of types (1) and (2) have difficulty in reliably preventing adhesions and do not show satisfactory performance due to the limited number of adhesions that can be prevented or problems with the affinity of the material itself to the body.

On the other hand, type (3) anti-adhesion agents including liposome-mediated non-steroidal anti-inflammatory agents, inhibitors of reactive oxygen species, retinoid derivatives, halofuginone, plasminogen, synthesis and secretion promoters of plasminogen activators, proteases produced by certain bacteria, and cyclopropyl amine compounds, cyclopropanoic acid amide compounds, serum anti-adhesion materials containing albumin, heparin, heparinized methionine, leucine, polyhydric alcohol, etc. are known, although their effectiveness is uncertain. (Patent Document 1).

### PRIOR ART DOCUMENTS

### Patent Documents

Patent Document 1: JP 2008-155014 A

Patent Document 2: JP 2006-231090 A

Patent Document 3: JP 2000-037450 A

Patent Document 4: JP 2010-213984 A

Patent Document 5: WO 2015/029892 A

### Non-Patent Documents

Non-patent document 1: Akira Fujishita, Yoshiyuki Yoshida, Tomoko Shimomura, Ayumi Matsumoto: “A general review of anti-adhesion methods and measures to prevent adhesions—with a focus on gynecology-related literature”, Obstetrics and Gynecology in Practice, Vol. 59, No. 8, pp. 1159-1167, 2010.

Non-patent document 2: Hisashi Sugihara: “Erythrocyte hemolysis induced by glycerol,” Clinical Hematology, Vol. 24, No. 8, pp. 1012-1019, 1983.

Non-patent document 3: T. Takeda, Y. Ishida, and M. Kawashima: “Experimental study using rats on glycerol enema solution and hemolysis,” Journal of the Japanese Society for Nursing Research, Vol. 26, No. 4, pp. 81-88, 2003.

Non-patent document 4: T. Takeda: “An Empirical Study Using Laboratory Animals on Hemolysis Induced by Glycerin Enema,” Journal of the Japanese Society of Nursing Technology, Vol. 5, No. 1, pp. 45-50, 2006.

### SUMMARY OF THE INVENTION

### Problem to be Solved by the Invention

As a result of investigating the problems of anti-adhesion membranes made of bioabsorbable materials, the inventor has found the following. As a result of detailed observation of the adverse effects of the conventional anti-adhesion membranes made of materials that are degraded and absorbed in vivo, the inventor found that in the process of degradation and absorption of the membranes in vivo, countless macrophages, which process foreign substances in vivo, migrate to the anti-adhesion membrane. In the process of adhesion prevention, macrophages, which process foreign substances in vivo, migrate in countless numbers and accumulate in huge numbers in the anti-adhesion membrane. They also found that a large number of fibroblasts and capillaries invade the anti-adhesion membrane as a result of macrophage activity.

It is well known that macrophages produce a large amount of cell-inducing factors as well as phagocytosing foreign substances by active migration. If the membrane is made of biodegradable and absorbable material of an area of about 10 to 20 cm square, with a thickness of about 0.1 mm, macrophages as small as 15 microns must phagocytose it, and a huge number of macrophages will be involved in the phagocytosis process. In this case, the amount of cell induction factor produced by macrophages would be extremely large.

In other words, a large number of macrophages each produce a large amount of cell-inducing factors that attract the fibroblasts involved in adhesion to the area where the anti-adhesion membrane has been placed to form connective tissue. Numerous capillaries also invade the area to follow the cells in order to supply nutrients necessary for cellular activities. Because these phenomena occur one after another, even though the anti-adhesion process has been completed, a large amount of fibroblasts form new connective tissue in the area during the process of degradation and absorption of the anti-adhesion membrane. The inventor has found that the formation of new connective tissue reduces the success rate of adhesion prevention.

### Means to Solve the Problem

In order to solve this inconvenience, the inventor decided to use a new concept to prevent adhesion, namely, to use a non-absorbable material that cannot be phagocytosed by macrophages. However, when a non-absorbable material is used, the body encapsulates the material and treats it as a foreign body. So-called encapsulation is known to occur. The encapsulated tissue can also become adherent tissue. Therefore, we have to carefully select the material to be used, prepare a non-cytotoxic, non-adhesive, non-irritating substrate (stealthy material), place the substrate as an adhesion inhibitor in the area where adhesion should not occur, and withdraw the adhesion inhibitor from the body as soon as possible after tissue healing is completed, that is, before the adhesion inhibitor is encapsulated. By developing an in vivo non-absorbable adhesion inhibitor suitable for this strategy, the inventor succeeded in completely preventing adhesion.

The problem of the present invention is that even though the bioabsorbable anti-adhesion membranes used in the conventional technology can prevent adhesion immediately after surgery, in the process of membrane absorption, the membranes are phagocytosed by countless macrophages, and new connective tissue is formed by fibroblasts attracted by the cell growth factor produced by the macrophages. In view of the fact that new connective tissue is formed and secondary adhesion occurs, the inventor provides an adhesion inhibitor that prevents adhesion by placing an adhesion inhibitor that is not degraded and absorbed in vivo near the wound, and then withdraws the adhesion inhibitor from the body without triggering the activity of macrophages after the healing of the wound is completed. In this way, the adhesion inhibitor can be withdrawn from the body without inducing macrophage activity.

It is necessary to pull out the adhesion inhibitor from the body as soon as possible after the adhesion prevented, and the timing is important. Therefore, the present invention aims to provide an anti-adhesion material that has an anti-adhesion part and a grasping part that can be removed from the body securely and safely.

As a result of intensive research to solve the above-mentioned problem, the inventor has decided to make the anti-adhesion material have an anti-adhesion part and a grasping part. The anti-adhesion part should have the property of not being recognized as a foreign body by the living body, the so-called stealthy property, and the property of not allowing cells and tissues to adhere to it. In addition, the grasping part needs to be designed in such a way that it can be used outside the body securely and safely without damaging the anti-adhesion part, and without entangling the biological tissue and pulling it out at the same time. The inventor has studied these issues intensively and has come up with the present invention.

In other words, according to the present invention, there is provided an anti-adhesion material as shown below.

[1] An anti-adhesion material, wherein at least a part of the anti-adhesion material is made of a non-biodegradable material and a contact angle of a surface to water of less than 7 degrees or more than 90 degrees.

[2] The anti-adhesion material according to [1], wherein at least the part of the anti-adhesion material is made of the non-degradable material, and the anti-adhesion material is to be removed from a body within 30 days after implantation.

[3] The anti-adhesion material according to [1] or [2], wherein the anti-adhesion material has an anti-adhesion part and a grasping part for withdrawal outside a body within 30 days after surgery.

[4] The anti-adhesion material according to [3], wherein the grasping part can be identified from a surrounding tissue by either ultrasonography, radiography, or palpation.

[5] The anti-adhesion material according to [3] or [4], wherein the grasping part is at least one type selected from a group consisting of a string, a membrane, a button, a wire, a fiber, a cloth, a mesh, and their combined state, or is a deformed part of the anti-adhesion part.

[6] The anti-adhesion material according to any one of [3] to [5], wherein at least a part of the grasping part and the anti-adhesion part is made of in vivo non-degradable material and has neither cytotoxicity nor cell adhesion.

[7] The anti-adhesion material according to any one of [3] to [6], wherein the anti-adhesion material binds or contains at least one selected from heparin, polyhydric alcohol, urokinase, tissue plasminogen, polyethylene glycol, polyvinyl alcohol, and vinylon.

[8] The anti-adhesion material according to any one of [3] to [7], wherein the anti-adhesion part and the grasping part are at least one selected from a group consisting of a membrane, a string, a tube, a rod, and a mesh, or a combination thereof.

[9] The anti-adhesion material according to any one of [3] to [8], wherein the anti-adhesion part is membranous and has convergent property, tissue slidabiliry, and tensile strength of greater of equal to 20 kPa (test method: JIS Z1702) for withdrawal outside the body through a small hole of 2 cm or less in diameter.

[10] The anti-adhesion part according to any one of [3] to [9], wherein the anti-adhesion part has a membrane shape and a peripheral portion of the anti-adhesion part has a membrane expansion and maintenance part such as a part with high rigidity compared to the central part of the anti-adhesion part, a part with a steel wire, a part with a tube, etc.

[11] The anti-adhesion part according to [10], wherein the membrane expansion and maintenance part has a steel wire made of a shape memory alloy, or a piano wire, or a wire having a similar stiffness and flexibility thereto.

[12] The anti-adhesion part according to [10], wherein a membrane expansion state is maintained by injecting a liquid into the tube arranged in the membrane expansion and maintenance part.

[13] The anti-adhesion part according to any one of [3] to [12], wherein the anti-adhesion part has a membrane shape and is inserted during surgery into any one of an abdominal cavity, a thoracic cavity, a pericardial cavity, or an intracranial space, and the grasping part is used by penetrating an abdominal wall, a thoracic wall, a skull, or the like and being fixed in a subcutaneous tissue just below a skin.

[14] The anti-adhesion part according to any one of [3] to [13], wherein the anti-adhesion part is inserted into any one of a tubular tissue such as a tear duct, a ureter, a urethra, a tendon sheath, etc., and the grasping part is fixed in a subcutaneous tissue just below a skin.

[15] Anti-adhesion part according to [13] or [14], wherein the anti-adhesion part inserted during surgery can be exposed and grasped by a small incision in the skin where the grasping part is located within a predetermined period of time after surgery, and can be pulled out of the body to prevent late adhesion.

### Effects of the Invention

The anti-adhesion material of the present invention completely prevents the formation of adherent tissue, which may occur in association with active healing activities by fibroblasts and other cells in the immediate postoperative surgical wound, by interposing an anti-adhesion material made of in vivo non-absorbable material to which cells do not adhere easily, and also prevents the formation of secondary adherent tissue induced by the activities of macrophages.

However, when such an in vivo non-absorbable material is present in the body, the body begins to surround it with connective tissue, which is called encapsulation activity, and the encapsulated tissue can also become adherent tissue, so it is essential to remove the in vivo non-absorbable material before this activity begins. Therefore, the anti-adhesion material is designed with a grasping part to facilitate the withdrawal of the in vivo non-absorbable material by grasping the grasping part, and the anti-adhesion part is removed from the body in a timely manner. This design provides permanent adhesion inhibition.

### BRIEF DESCRIPTION OF THE DRAWINGS

FIG. 1 is a schematic diagram of anti-adhesion material of an embodiment of the present invention.

FIG. 2 is a conceptual diagram showing the use of the anti-adhesion material inserted into the abdominal cavity.

### EMBODIMENT FOR CARRYING OUT THE INVENTION

The anti-adhesion material of the present invention belongs to (1) a material that is inserted as a physical barrier to prevent adhesion, according to the classification described in paragraph 0005. However, the anti-adhesion material used is designed to be used as a physical barrier inside the body to prevent adhesion only during the period immediately after surgery when adhesion is likely to occur, and to be removed from the body when the healing of the surgical wound inside the body is completed. Therefore, since it is of utmost importance to take it out of the body, the anti-adhesion material has an anti-adhesion part that securely prevents adhesion and a grasping part for taking it out.

The grasping part is responsible for withdrawing the anti-adhesion material from the abdominal cavity or thoracic cavity after it is inserted into the abdominal cavity or thoracic cavity, fixing it in the subcutaneous tissue, and grasping it through a skin incision within a predetermined period of time after surgery, preferably within 30 days, and withdrawing the anti-adhesion material from the body.

Since the grasping part is implanted in the subcutaneous tissue, the position of the grasping part is confirmed by palpation after surgery, and the part is removed through a skin incision. In case it is impossible to confirm the position of the grasping part by palpation due to a large amount of subcutaneous fatty tissue depending on the patient's constitution, the grasping part should have the property of being able to be confirmed by using an ultrasound diagnostic device or radiography device. In other words, the grasping part should have a different ultrasonic reflection performance from that of the surrounding tissue, or should have a part of the material that is radiopaque.

The shape of the grasping part can be a string, membrane, button, wire, fiber, cloth, mesh, or a combination of these. The anti-adhesion material must be able to be pulled out without fail and must be strong enough not to be torn off when grasped, so protruding and deforming a part of the aforementioned anti-adhesion part to make it easier to grasp is acceptable as long as it is strong enough and the anti-adhesion part is not damaged by grasping.

The grasping part should not have cytotoxicity or cell adhesiveness, as it will be difficult to pull out if it is buried in the surrounding tissue and entangled. In the same way, the anti-adhesion part must not be cytotoxic or cell-adhesive, and must be stealthy so that it can be accepted by the body without trouble.

Stealthy materials in vivo used in the anti-adhesion part and the grasping part include fluoropolymers, polyester polymers, polyolefin polymers, polyamide polymers, polyethylene polymers, silicone polymers, polycarbonate polymers, polyvinyl vinylone, rayon, polyvinyl alcohol, polyethylene glycol, gelatin, collagen, chitin, partially deacetylated chitin, chitosan, hyaluronic acid, carboxymethyl cellulose, acrylic polymers, graft polymers thereof, derivatives thereof, cross-linkers thereof, salts thereof. It is preferable to consist of at least one type selected from the group consisting of the above, or hybrids, etc.

The shape of the anti-adhesion part can be a membrane, string, tube, rod, mesh, or a combination of these shapes. The shape can be prepared according to the part to be used.

When the anti-adhesion material is inserted into the abdominal cavity or the thoracic cavity, the main purpose of this invention is to prevent macrophages from being active, and for this purpose, it is necessary to use a material that is not phagocytosed by macrophages, that is, not broken down and absorbed in vivo. For this purpose, it is necessary to use a material that is not phagocytosed by macrophages, i.e., not broken down and absorbed in vivo. The grasping part is also mainly made of non-bioabsorbable material, since it cannot be grasped if it is absorbed.

However, if the in vivo non-absorbable material used for the anti-adhesion part is not highly capable of blocking cell adhesion, or if it must be used for patients who are prone to adhesions, it is effective to include a small amount of cell adhesion blocking auxiliary agent in the anti-adhesion part, or to make it intertwined with it to enhance the adhesion blocking effect. For example, glycerin, one of the polyhydric alcohols, helps to prevent cell adhesion. However, cell adhesion inhibitors such as glycerin diffuse in vivo and are processed by hydrolysis and enzymes, and do not mobilize macrophages. Therefore, it is recommended to use cell adhesion inhibitors such as heparin, polyhydric alcohol, urokinase, tissue plasminogen, polyethylene glycol, polyvinyl alcohol, vinylon, etc., which are easily diffused in vivo and do not induce macrophage activity.

When the anti-adhesion part is pulled out of the body by the grasping part, it is pulled out through a narrow tissue hole of less than 2 cm in diameter. When the anti-adhesion part is made of a membrane, it needs to have convergence and tissue slidability in order to pass through the small hole. The most important thing is to avoid the membrane from being torn off and remaining in the body when it is pulled out. If the anti-adhesion part is too thick, it will be difficult to pull out. Therefore, a thin membrane must be used, but the thinner the membrane, the weaker it becomes in terms of strength, so a tensile strength of 20 kPa (test method: JIS Z1702) or higher is required.

Therefore, one of the innovations of this invention is to increase the strength of the thin film by adding a hybrid structure with fibers and meshes.

The surface of the anti-adhesion part should be hydrophilic, with a surface contact angle of 7 degrees or less. Considering that the contact angle of water to glass is about 8 degrees, the material should be more hydrophilic than the glass surface. In the case of a surface that is more hydrophilic than the glass surface, it has a slight slippery property when it comes in contact with water, but it is known that there is little cell adhesion on such a slippery surface. It is recommended that the surface be hydrophilic with a contact angle to water of greater than 0 degrees and less than 7 degrees, preferably greater than 0 degrees and less than 6 degrees.

On the other hand, it is also known that cells do not adhere easily to the slippery surface of hydrophobic materials. Therefore, it is preferable that the surface property of the anti-adhesion part is hydrophobic with a surface contact angle to water of 90 degrees or more. According to general data, the contact angle of nylon is about 70 degrees, Polyvinyl-chloride is 87 degrees, polystyrene is 91 degrees, polytetrafluoroethylene is 108 degrees, polyethylene is 94 degrees, and paraffin is 108-116 degrees. In this study, in order to prevent cell adhesion for a certain period of time after surgery, it is recommended that the contact angle of the hydrophobic surface be as large as polystyrene, specifically, the contact angle of the surface to water should be between 90 degrees and 180 degrees, preferably between 90 degrees and 170 degrees.

If the anti-adhesion part is a thin membrane, it is necessary to keep the membrane expanded for about one week after the surgery while the surgical wound heals. In order to do so, the peripheral portion of the anti-adhesion part should have a more rigid and flexible part than the central part, or steel wires should be arranged, or thin shape memory alloys, piano wires, or other wires with similar rigidity should be arranged, or thin tubes should be arranged and fluids such as physiological saline should be injected into the tubes under pressure. It is characterized by having a membrane expansion and maintenance part, such as a thin tube with a liquid such as physiological saline pressure injected into the tube.

When removing the anti-adhesion part with such an expansion and maintenance part from the body, the shape memory alloy or piano wire should be removed first, or a liquid such as physiological saline that has been injected into the thin tube should be drained to soften the area around the membrane and facilitate removal of the anti-adhesion part from the thin tissue hole.

The anti-adhesion material with a grasping part having the structure described above is preferably used in a way that it is inserted during surgery into the abdominal cavity, thoracic cavity, pericardial cavity, or intracranial space, and the former grasping part penetrates the abdominal wall, chest wall, or skull, and is fixed in the subcutaneous tissue just under the skin. It is desirable to have properties that are suitable for such usage.

When the above-mentioned anti-adhesion material is in the shape of a tube, string, or rod, it is preferable to use it in such a way that it is inserted into tubular tissues such as tear ducts, ureters, urethra, tendon sheaths, etc., and the grasping part is fixed in the subcutaneous tissue just below the skin, and it is preferable to provide it with characteristics suitable for such usage.

With regard to the withdrawal of the anti-adhesion material from the body within a certain period of time after surgery, the timing varies depending on the part of the body where the adhesion inhibitor is to be used, as well as the patient's age, gender, nutritional status, and presence of underlying diseases. For example, in healthy children, cellular activity is active and wound healing is rapid, so it is recommended that the abdomen be treated within 5 days after surgery. In the case of abdominal surgery, the device can be removed after the fifth postoperative day, and it is preferable to remove the device within two weeks because unexpected adhesions due to encapsulation may occur after two weeks. On the other hand, patients with poor nutritional status, such as the elderly or those with diabetes, tend to have delayed healing, so it is preferable to remove the device at least one week after surgery, preferably 10 days after surgery. However, it is not advisable to leave the device in place for more than 30 days, as it may cause unexpected adhesions due to encapsulation. Therefore, it is advisable to remove the anti-adhesion material within a predetermined period of time after the surgery, at the latest within 30 days.

The following figure illustrates the anti-adhesion material of the present invention. The figure shown here is only a conceptual diagram, and the invention is not restricted to the shape shown in this figure.

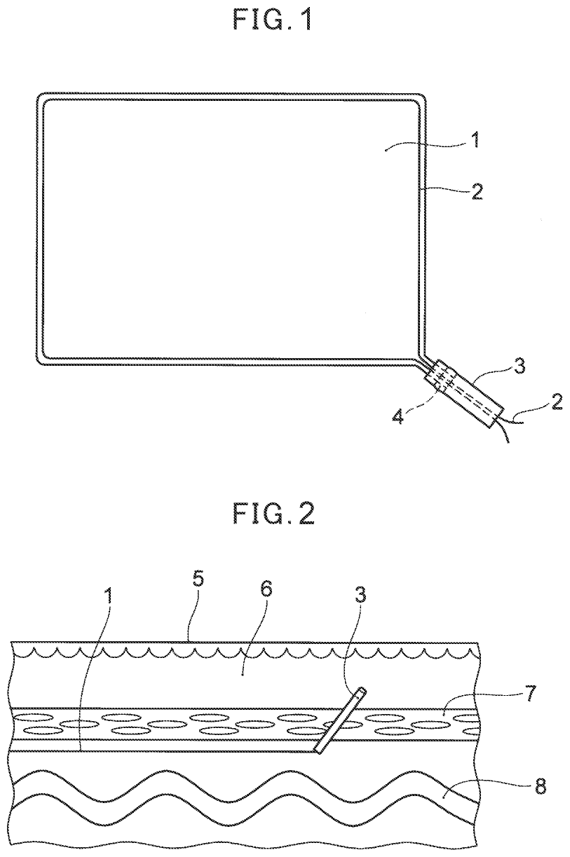

In FIG. 1, the anti-adhesion part is shaped as a membrane, shown as 1, and the shape memory alloy wire placed around the membrane is shown as 2, which maintains the expansion of the membrane. The grasping part is indicated by 3. The membrane 1 can be pulled out of the body by grasping the grasping part 3 through the tissue tunnel, which had left around the grasping part 3. The fixation point between the membrane and the grasping part is indicated by 4, which prevents the membrane 1 from separating when the grasping part 3 is pulled out. The shape-memory alloy wire 2 is also connected to the grasping part 3 to prevent excessive tension from being applied to the membrane 1 when the grasping part 3 is pulled, so that the membrane 1 does not tear off.

FIG. 2 shows a conceptual diagram of the use of the anti-adhesion material of the present invention inserted into the abdominal cavity. The figure shown here is just one conceptual diagram, and the invention is not restricted to the shape shown in this figure.

In FIG. 2, 1 is the anti-adhesion part of the anti-adhesion material of the present invention, which is placed in the abdominal cavity. The abdominal cavity contains the intestinal tract as shown at 8, the skin at 5, and the muscular layer of the abdominal wall at 7. The anti-adhesion material 1 is attached to a grasping part as shown at 3, and the end of the grasping part 3 is fixed in the subcutaneous tissue as shown at 6. Specifically, the grasping part 3 is sewn into the subcutaneous tissue 6 with sutures to prevent it from being dragged into the abdominal cavity. After a certain period of time has elapsed after the surgery, a small skin incision is made on the skin of the patient's body, and the grasping part of 3 is grasped and pulled out of the peritoneal cavity, so that the anti-adhesion material 1 does not remain in the body and does not cause subsequent macrophage activity. This type of use is recommended in the present invention.

### EXAMPLES

Next, the present invention will be described in more detail with examples. The present invention is not limited by these examples.

### Example 1

We selected polyester, polypropylene, and rayon as representative examples among the materials recommended for use in anti-adhesion part and grasping part, and evaluated whether macrophages would accumulate when they were embedded in vivo. The sample used was a commercially available wet tissue paper (wet tissue, manufactured by Lion Corporation). The wet tissue paper contains rayon fiber, polyester fiber and polypropylene fiber.

Therefore, if the wet tissue paper material is used, rayon, polyester, and polypropylene will be evaluated.

A commercially available wet tissue paper was washed well in running water to remove water-soluble adhesions, then washed in 70% ethanol to remove adhesions soluble in organic solvents, air-dried, and sterilized by low-temperature EOG to make a test sample. Then, 2×2 cm sample pieces were inserted into the subcutaneous tissue of rats and collected after 1 week, 2 weeks, 3 weeks, and 4 weeks. They were embedded in the hydrophilic resin Technovit (Kulzer Co., Germany), and sections of 3 microns in thickness were made with a glass knife. The sections were stained with hematoxylin and eosin and observed under an optical microscope at 100-400x. As a result, no accumulation of macrophages was observed near the polyester, polypropylene, and rayon fibers from 1 to 4 weeks after implantation. However, at the fourth week, fibroblasts were observed to form a capsule around each fiber. As a result, it was found that the evaluated polyester, polypropylene, and rayon fibers did not accumulate macrophages after implantation, and at the same time, encapsulation occurred around the fibers when they were left implanted for about four weeks.

### Comparative Example 1

An anti-adhesion membrane was prepared using sodium hyaluronate, the main ingredient of Seprafilm, which is currently available commercially and used in clinical practice. First, a 1% solution of sodium hyaluronate was prepared, which was poured into a stainless-steel Petri dish and air-dried to produce a thin film of sodium hyaluronate with a thickness of 40 microns. The film was then insolubilized with acetic anhydride, washed thoroughly in running water, air-dried, and sterilized by EOG to make a test sample. The method of insolubilizing sodium hyaluronate using acetic anhydride was in accordance with the method described in Patent Document 5.

Sample pieces of 2×2 cm were then inserted into the subcutaneous tissue of rats and collected after 1, 2, 3, and 4 weeks, embedded in hydrophilic resin Technovit, and sections of 3 microns in thickness were prepared with a glass knife, stained with hematoxylin and eosin, and observed under an optical microscope at 100-400x. As a result, one week after implantation, the samples were slightly swollen, and macrophages were observed to accumulate around them. In the second week after implantation, the swelling of the sample and the accumulation of macrophages became more pronounced, and in the third week after implantation, the swelling of the sample and the accumulation of macrophages became even more pronounced, and macrophage invasion into the sample was also observed. At the fourth week after implantation, the swelling of the samples became more pronounced, and at the same time, numerous macrophages invaded the samples and actively phagocytosed the hyaluronan in the samples. At the same time, the accumulation of macrophages became more pronounced, and the active invasion of macrophages into the sample was observed. In addition, numerous fibroblasts accumulated around the sample and surrounded it, and many collagen fibers were observed, indicating the formation of cellular fibrous connective tissue. As a result, it was found that hyaluronic acid, which is a representative of bioresorbable materials, swells after implantation and begins to dissolve in vivo, at the same time accumulating countless macrophages and inducing phagocytosis, and that if this state continues, cellular fibrous connective tissue is formed around the sample, which is the source of adherent tissue. It was found that the macrophages accumulate and induce phagocytosis.

### Example 2

It is generally known that foreign substances do not adhere easily to highly hydrophobic base materials such as Teflon (registered trademark). However, there is little data on how hydrophobic the material should be in order to prevent cell adhesion. In particular, in this invention, we examined materials that can prevent cell adhesion for a certain period of time, at least one week, in vivo. The cells used in the evaluation were commercially available human dermal fibroblast, adult (HDFa). The cells were cultured on polystyrene petri dishes according to general cell culture techniques. The material to be evaluated was placed on a polystyrene Petri dish, and cells were seeded on it. After seeding, cells were observed on the material every day, and a syringe was used to sprinkle the cell culture solution on the cell surface in the form of a water jet to examine the ease of cell detachment from the material.

The materials used were nylon, PVC, polystyrene, polytetrafluoroethylene, polyethylene, and paraffin. As a result, cells adhere easily to the nylon surface and are difficult to peel off once attached. On the other hand, cells do not adhere to paraffin. The cells adhered to polystyrene but peeled off easily, while the cells adhered to polyethylene but peeled off easily, and this tendency was more pronounced than for polystyrene. Cells do not adhere to polytetrafluoroethylene. Polyvinyl chloride (PVC) had cells that adhered and peeled off easily. These results suggest that PVC and nylon are not sufficient to prevent cell adhesion in the first week after surgery. The results showed that PVC and nylon were not good enough to prevent cell adhesion for about one week after the surgery, and that a material with a property that prevents cell adhesion such as polystyrene or polyethylene was preferable. The contact angles of the individual materials used against water are as follows. In other words, the contact angle of nylon is about 70 degrees, PVC is 87 degrees, polystyrene is 91 degrees, polytetrafluoroethylene is 108 degrees, polyethylene is 94 degrees, and paraffin is 108 to 116 degrees. In order to prevent the adhesion of cells for a certain period of time after surgery, it is preferable to have a hydrophobic surface with a contact angle as large as polystyrene. In other words, a hydrophobic material with a contact angle of 90 degrees or more was found to be preferable.

[Embodiment 3]

On the other hand, it is generally known that it is difficult for cells to adhere to highly hydrophilic substrates such as agar.

Therefore, in this invention in particular, we examined a hydrophilic material that can prevent cell adhesion for a certain period of time in vivo, at least about one week. As in Example 1, the cells used in the evaluation were commercially available human dermal fibroblast, adult (HDFa). The cells were cultured on polystyrene Petri dishes according to general cell culture techniques. The material to be evaluated was placed on a polystyrene Petri dish, and cells were seeded on it. After seeding, cells were observed on the material every day, and a syringe was used to sprinkle the cell culture solution on the cell surface in the form of a water jet to examine the ease of cell detachment from the material.

The materials used were glass, agar, gelatin, polyethylene glycol-grafted vinyl chloride, vinylon, and polyvinyl alcohol cross-linked material spread on wet tissue paper. As a result, cells adhere easily to the glass surface and are difficult to peel off once attached. On the other hand, cells do not adhere to polyethylene glycol grafted vinyl chloride and vinylon. On the other hand, cells did not adhere to polyvinyl alcohol grafted vinyl chloride and vinylon. From these results, it was found that glass was not sufficient to prevent cell adhesion during the first week after surgery, and that materials with properties that prevent cell adhesion, such as polyethylene glycol grafted vinyl chloride, vinylon polystyrene, and polyethylene, were preferable. The contact angles to water of the individual materials used are as follows. In other words, the contact angle of glass was about 8 degrees, that of vinylon and polyvinyl alcohol cross-linked materials was 2 to 3 degrees, that of polyethylene glycol grafted vinyl chloride was 1 degree, and that of agar and gelatin was less than 5 degrees, although the exact values could not be determined due to the preparation conditions. In order to prevent the adhesion of cells for a certain period of time after surgery, it is preferable to have a hydrophilic surface with a smaller contact angle than a glass surface. In other words, a hydrophilic material with a contact angle of less than 7 degrees was found to be preferable.

[Embodiment 4]

The commercially available wet tissue paper was washed and dried according to the method described in Example 1. The wet tissue paper contains extremely fine rayon fibers, and polyester and polypropylene fibers are intertwined to maintain strength, making it hydrophilic, hydrophobic, and strong. The size was only slightly smaller than A4. The dried wet tissue paper substrate was soaked in a 3% polyvinyl alcohol solution and air-dried. A polyvinyl alcohol with a saponification rate of 98% and a degree of polymerization of 1000 was selected. Next, the air-dried wet tissue paper substrate soaked with polyvinyl alcohol was exposed to formalin vapor to insolubilize the polyvinyl alcohol, and then washed thoroughly under running water and air-dried to form a membrane of the in vivo non-absorbable material of the anti-adhesion membrane. The contact angle of the surface of this membrane to water was measured with a DMo-501 contact angle meter manufactured by Kyowa Interface Science Co. Ltd, and was found to be 2 degrees. This membrane is referred to as A membrane.

In the case of the A membrane, a steel wire of 0.4 mm in diameter made of Nitinol alloy was sewn to one end of the A membrane to spread the membrane. Nitinol alloy steel wire was pulled out from one of the four corners of the A membrane, and a silicone tube of 8 mm in outer diameter and 5 cm in length was placed on this part to fix the A membrane and the Nitinol alloy steel wire to form the grasping part. The prototype anti-adhesion material I made in this way was sterilized by low-temperature EOG.

The abdominal cavity of an adult dog was opened through a midline incision in the abdomen under general anesthesia, and the prototype anti-adhesion material I was spread just below the surgical wound, and the grasping part was fixed by penetrating the abdominal wall muscle layer near the liver and inserting its tip into the subcutaneous tissue. The surgical wound in the abdomen was closed and the operation was completed.

One week after the surgery, the dog was anesthetized again, and when the abdomen was observed with an ultrasound machine, the anti-adhesion membrane and Nitinol steel wire could be seen, and the X-ray also showed that the Nitinol steel wire looked like a ring and that the membrane was spreading. Then, the grasping part was confirmed by palpation, the grasping part was picked up through a skin incision of about 2 cm, the grasping part was grasped with Kocher's forceps, and the prototype anti-adhesion material I was pulled out. The removal was easy, and it was confirmed that the membrane was completely removed from the body without any tears or damage.

Three weeks later, the experimental dog was again anesthetized and the abdomen was observed with an ultrasound device, and the movement of the intestinal tract in the abdominal cavity and the respiratory movement confirmed that there were no adhesions. For further confirmation, the abdomen was opened through a midline incision to check for adhesions, and adhesions of the intestinal tract and reticular tissue to the surgical wound were completely prevented.

### Example 5

An experiment was conducted in which the prototype anti-adhesion material I was inserted into the left thoracic cavity of an experimental dog under general anesthesia. As a specific surgical method, the animal was placed in a right lateral position, the left seventh intercostal space of the pleural cavity was opened, and the anti-adhesion material I was spread just below the surgical wound. The grasping part was fixed in place by penetrating the muscular layer of the chest wall near the fourth intercostal space and inserting their tips into the subcutaneous tissue. The surgical wound in the chest was closed and the operation was completed.

One week after surgery, the same x-ray and ultrasound examinations were performed as those performed on the abdomen, and it was confirmed that the prototype anti-adhesion material I maintained its spread in the thoracic cavity. Then, the grasping part was confirmed by palpation of the skin near the fourth intercostal space of the experimental dog, and 2 cm of skin was cut at the site to expose the grasping part, and the prototype anti-adhesion material I was withdrawn by grasping the tip. The membrane was not damaged and could be easily withdrawn. Then, three weeks after the surgery, the respiratory movement of the lung was observed again under general anesthesia using a chest ultrasound system, and it was confirmed that there was no adhesion between the lung and the wall of the pleural cavity. Then, the eighth intercostal space was opened and the thoracic cavity was visually examined, and no adhesions were observed between the lungs and the pleural wall.

### Example 6

The prototype anti-adhesion material I was soaked in glycerin and then sterilized by low-temperature EOG. The size of the membrane was 10 cm square, and the length of the silicone tube of the grasping part was 15 cm. This membrane is called Prototype anti-adhesion membrane II. The contact angle of this membrane to water was 1 degree.

Under general anesthesia, the dog's chest was opened at the seventh intercostal space of the left chest, and the pericardium was cut open to expose the heart. The prototype anti-adhesion membrane II was then placed in direct contact with the heart surface. The incision of the pericardium was closed, and the silicone tube of the grasping part was fixed in the subcutaneous tissue of the abdominal wall by penetrating the diaphragm.

One week after the surgery, the same x-ray and ultrasound examinations were performed as those performed for the abdomen and chest, and it was confirmed that the prototype anti-adhesion membrane II maintained its spread around the heart. Then, the grasping part was confirmed by palpation of the skin on the abdominal wall of the experimental dog, and 2 cm of skin was cut at the site to expose the grasping part, and the prototype anti-adhesion membrane II was pulled out by grasping the tip. The membrane was not damaged and could be easily withdrawn. Three weeks after the surgery, the seventh intercostal space was opened again under general anesthesia and the thoracic cavity was visually examined, and there were no adhesions between the pericardium and the heart.

[Embodiment 7]

In Example 3, the wet tissue paper was used as the base material, but the effect of the invention was verified using a hydrophobic membrane in this example. Specifically, a Naflon membrane manufactured by Nichias Corporation, with a thickness of 0.05 mm, was used. The contact angle of the surface of this membrane to water was measured with a DMo-501 contact angle meter manufactured by Kyowa Interface Science Co. Ltd, and was found to be 95 degrees. In other words, it is an anti-adhesion material using an extremely hydrophobic base material. This film is called B membrane.

A piano wire with a wire diameter of 0.25 mm was sewn to one end of the B membrane to spread the membrane. Next, a piano wire was pulled out from one of the four corners of the B membrane, and a silicone tube with an outer diameter of 8 mm and a length of 5 cm was placed on this part to fix the B membrane piano wire as a grasping part. The prototype made in this way was used as the prototype anti-adhesion membrane III, which was sterilized by autoclaving.

The adhesion inhibition effect was confirmed in the abdominal cavity of an adult dog using the prototype anti-adhesion membrane III in the same surgical procedure as shown in Example 3, and the same results as in Example 3 were obtained. As a result, it became clear that the prototype anti-adhesion membrane III was also capable of preventing adhesions in the present invention.

### Comparative Example 2

The made A membrane was inserted into the abdominal cavity of an adult dog in the same way as in Example 1. The membrane was only placed on the intestine and could not be withdrawn because there was no grasping part. Three weeks after the surgery, the experimental dog was opened under general anesthesia, and the membrane was stuck in the pelvic cavity and had not spread. In the abdominal cavity, there were no intestinal adhesions, but the greater omentum was adhered to the surgical wound. As a result, it was found that without the grasping part, the membrane could not be pulled out, and there was also a problem with the fixation of the membrane in the abdominal cavity, causing it to move downward in the abdominal cavity. In addition, since no shape memory alloy steel wire was used to expand the membrane, it was found that the membrane had shrunk and could not cover the surgical wound.

### Comparative Example 3

An adult dog under general anesthesia had its chest opened at the left seventh intercostal space and the surgical wound was closed.

Three weeks later, the dog underwent general anesthesia and the chest was opened at the seventh intercostal space again, but it could not be opened because the lung tissue was firmly adhered to the wound. When the chest was opened at the 9th intercostal space and the 7th intercostal space was observed, it was found that the lung tissue was tightly adhered to the surgical wound. As a result, it was found that the lung tissue was extremely prone to adhesion without the use of adhesion inhibitors, and that some adhesion inhibitors are essential for open chest surgery.

### Comparative Example 4

An adult dog under general anesthesia had its chest opened at the left seventh intercostal space and the pericardium was opened to expose the heart. Next, the pericardium was closed, and then the surgical wound in the chest wall was closed. Three weeks later, under general anesthesia, the chest was opened again at the seventh intercostal space, but as in the comparative example, the lung was adherent to the wound and could not be opened. When the chest was opened at the 9th intercostal space and the 7th intercostal space was observed, it was found that the lung tissue was tightly adhered to the surgical wound. When the pericardium was opened further, the pericardium was adhered to the heart surface. As a result, it was found that both the lung tissue and the heart surface were extremely prone to adhesion without the use of adhesion inhibitors, and that some adhesion inhibitors were essential for open heart surgery and cardiac surgery.

### Example 8

The A membrane fabricated in Example 3 was soaked in glycerin to form a membrane strip of 5 cm in length and 1 cm in width, and the grasping part was fabricated using an e-PTFE suture on one side of the membrane. The contact angle of the membrane surface to water was 1 degree. This is the prototype anti-adhesion membrane IV.

A chicken was anesthetized and the back of the right leg was opened over a 6 cm length to expose the tendon that runs through the middle of the leg. The tendon was then wrapped with the prototype anti-adhesion membrane IV, and the grasping part made with e-PTFE suture was pulled out from the edge of the surgical wound and fixed outside the skin.

Looking at the condition of the chicken's legs after the surgery, especially the spread of the toes, we found that the chicken was limping immediately after the surgery and the next day, but no abnormalities were observed after that. Therefore, on the seventh day after the surgery, we made a hole of about 5 mm to the tendon by pulling the grasping part made with e-PTFE suture, and then pulled out the prototype anti-adhesion membrane IV by pulling the grasping part made with e-PTFE suture.

When we observed the movement of the chicken's legs, especially the spread of the toes, until three weeks after the surgery, there was no change in the spread of the toes on either side and no abnormality was observed at all. Therefore, when the chicken was again subjected to general anesthesia and the tendon of the leg were opened, there were no adhesions around the tendon.

### Comparative Example 5

A chicken was anesthetized in the same manner as in Example 8, and the back of the leg was opened over a length of 6 cm to expose a tendon running through the middle of the leg. The tendon was then wrapped with the prototype anti-adhesion membrane IV. At this time, the grasping part made with e-PTFE suture was removed, and the anti-adhesion material without the grasping part was implanted.

When we looked at the condition of the chicken's leg after the surgery, especially the spread of their toes, we found that it was limping immediately after the surgery and the next day, but no abnormalities were observed after that. After three weeks of observation, the spread of the toes became worse, and the toes of the left leg, which had not been operated on, spread and walked, but the toes of the right leg did not spread sufficiently, and the chicken began to waddle slightly.

Therefore, after 5 weeks, the chicken was again subjected to general anesthesia and the leg tendon area was opened, and the anti-adhesion membrane was entangled around the tendon, and capsule formation was observed with connective tissue covering the surrounding area. As a result, the fabricated prototype anti-adhesion membrane IV temporarily prevented adhesion, but when left for a long period of time, capsule tissue formed around it and adhesion by the capsule tissue was found to occur. As a result, it was found that the anti-adhesion membrane IV, which was not absorbed in vivo, needed to be removed after a certain period of time after the surgery, and a grasping part was necessary to pull it out.

### Comparative Example 6

A chicken was anesthetized in the same manner as in Example 8, and the back of the leg was opened over a length of 6 cm to expose the tendon that runs through the middle of the leg. Then, the surgical wound was closed without touching the tendon. In other words, no anti-adhesion material was used.

When we observed the condition of the chicken's legs after the surgery, especially the spread of the toes, we found that the toes did not spread sufficiently, and they were limping immediately after the surgery and the next day, but even after that, it was limping and there was no improvement. Three weeks after the surgery, the spread of the toes became worse, and the toes on the left leg, which had not undergone surgery, spread and walked, but the toes on the right leg did not spread sufficiently, and the chicken constantly limped.

Therefore, after five weeks, the chickens were again subjected to general anesthesia and the tendon area of the leg was opened, and an adhesion tissue was formed around the tendon, restricting the movement of the tendon. This result showed that adhesions were likely to form around the tendon if no anti-adhesion material was used, and that the formation of adhesion tissue restricted the movement of the tendon, indicating that some means of preventing adhesion, namely anti-adhesion material, was necessary for tendon surgery.

### INDUSTRIAL APPLICABILITY

The anti-adhesion material of the present invention can safely and reliably prevent postoperative adhesions in various tissues and sites.

1 ANTI-ADHESION PART

2 WIRE

3 GRASPING PART

4 PLACE TO FIX THE MEMBRANE OF THE ANTI-ADHESION PART AND THE GRASPING PART

5 SKIN

6 SUBCUTANEOUS TISSUE

7 MUSCLE LAYER

8 INTESTINE

1. An anti-adhesion material, wherein at least a part of the anti-adhesion material is made of a non-biodegradable material and a contact angle of a surface to water is less than or equal to 7 degrees or greater than or equal to 90 degrees.

2. The anti-adhesion material as claimed in claim 1, wherein at least the part of the anti-adhesion material is made of the non-biodegradable material, and the anti-adhesion material is to be removed from a body within 30 days after implantation.

3. The anti-adhesion material as claimed in claim 1, wherein the anti-adhesion material has an anti-adhesion part and a grasping part for withdrawal outside a body within 30 days after surgery.

4. The anti-adhesion material as claimed in claim 3, wherein the grasping part can be identified from a surrounding tissue by either ultrasonography, radiography, or palpation.

5. The anti-adhesion material as claimed in claim 3, wherein the grasping part is at least one type selected from a group consisting of a string, a membrane, a button, a wire, a fiber, a cloth, a mesh, and their combined state, or is a deformed part of the anti-adhesion part.

6. The anti-adhesion material as claimed in claim 3, wherein at least a part of the grasping part and the anti-adhesion part is made of in vivo non-biodegradable material and has neither cytotoxicity nor cell adhesion.

7. The anti-adhesion material as claimed in claim 3, wherein the anti-adhesion part binds or contains at least one selected from heparin, polyhydric alcohol, urokinase, tissue plasminogen, polyethylene glycol, polyvinyl alcohol, and vinylon.

8. The anti-adhesion material as claimed in claim 3, wherein the anti-adhesion part and the grasping part are at least one selected from a group consisting of a membrane, a string, a tube, a rod, and a mesh, or a combination thereof.

9. The anti-adhesion material as claimed in claim 3, wherein the anti-adhesion part is membranous and has convergent property, tissue slidability, and tensile strength of greater than or equal to 20 kPa (test method: JIS Z1702) for withdrawal outside the body through a small hole of 2 cm or less in diameter.

10. The anti-adhesion material as claimed in claim 3, wherein the anti-adhesion part has a membrane shape and a peripheral portion of the anti-adhesion part has a membrane expansion and maintenance part such as a part with high rigidity compared to the central part of the anti-adhesion part, a part with a steel wire, a part with a tube, etc.

11. The anti-adhesion material as claimed in claim 10, wherein the membrane expansion and maintenance part has a steel wire made of a shape memory alloy, or a piano wire, or a wire having a similar stiffness and flexibility thereto.

12. The anti-adhesion material as claimed in claim 10, wherein a membrane expansion state is maintained by injecting a liquid into a tube arranged in the membrane expansion and maintenance part.

13. The anti-adhesion material as claimed in claim 3, wherein the anti-adhesion part has a membrane shape and is inserted during surgery into any one of an abdominal cavity, a thoracic cavity, a pericardial cavity, or an intracranial space, and the grasping part is used by penetrating an abdominal wall, a thoracic wall, a skull, or the like and being fixed in a subcutaneous tissue just below a skin.

14. The anti-adhesion material as claimed in claim 3, wherein the anti-adhesion part is inserted into any one of a tubular tissue such as a tear duct, a ureter, a urethra, a tendon sheath, etc., and the grasping part is fixed in a subcutaneous tissue just below a skin.

15. The anti-adhesion material as claimed in claim 13, wherein the anti-adhesion part inserted during surgery can be exposed and grasped by a small incision in the skin where the grasping part is located within a predetermined period of time after surgery, and can be pulled out of the body to prevent late adhesion.

16. The anti-adhesion material as claimed in claim 14, wherein the anti-adhesion part inserted during surgery can be exposed and grasped by a small incision in the skin where the grasping part is located within a predetermined period of time after surgery, and can be pulled out of the body to prevent late adhesion.
